(19) United States
(12) Patent Application Publication
(10) Pub. No.: US 2007/0083173 A1
(43) Pub. Date: Apr. 12, 2007

(54) Absorbent article featuring a temperature change member

(75) Inventor: Christopher P. Olson, Neenah, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/246,414

(22) Filed: Oct. 7, 2005

(51) Int. Cl. A61F 13/15

(52) U.S. Cl. 604/361

(57) ABSTRACT

Disclosed is an absorbent article including a temperature change member. The temperature change member includes temperature change material and a first folded region.

### BACKGROUND OF THE INVENTION

The present invention relates to absorbent articles that include a temperature change member. More specifically, the invention relates to an absorbent article such as training pants that provides the wearer with a noticeable temperature change sensation upon urination.

Absorbent articles such as disposable diapers and training pants are useful to absorb and contain body wastes. These products have developed to the extent that urine is quickly drawn and retained away from the wearer's skin so that the wearer remains relatively dry and comfortable. Although this improved performance enhances wearer dryness and comfort, it can reduce the wearer's ability to notice or recognize when urination occurs, especially if the wearer's attention is distracted by an activity. This is not conducive to toilet training because an important step in the early stages of toilet training is the ability to recognize when urination occurs. In an attempt to enhance a child's recognition of when urination occurs, training pants have been designed with temperature change members that provide a temperature change sensation upon urination.

Unfortunately, in certain circumstances, such temperature change members may not be completely satisfactory. For example, articles can, in certain circumstances, deform during use, particularly between the wearer's legs. As such, an article including a temperature change member may shift position and be less effective at alerting a wearer of urination if it is also spaced from the skin of the wearer.

Thus, there is a need for an absorbent article with a temperature change member that is capable of keeping at least a portion of the member close to the wearer in use. Further, there is a need for an absorbent article that is more likely to alert a wearer to urination even after the article has been deformed between the wearer's legs in use.

### SUMMARY OF THE INVENTION

In one aspect, the present invention is directed to an absorbent article defining an article first side edge and an article second side edge opposite the article first side edge and a pair of end edges. The absorbent article includes a liquid impermeable outercover, an absorbent body disposed on the outercover, and a temperature change member disposed with the absorbent body. The temperature change member includes a temperature change member first surface and a temperature change member second surface opposite the temperature change member first surface. The temperature change member also includes a temperature change material disposed between the first surface and the second surface where the temperature change first surface is in an overlaid relationship with itself to define a first folded region. The temperature change member also provides the article with a temperature change of at least 5 degrees C., as determined by the temperature change test described herein.

In another aspect, the present invention is directed to an absorbent article defining an article first side edge and an article second side edge opposite the article first side edge and a pair of end edges. The absorbent article includes a liquid impermeable outercover, an absorbent body disposed on the outercover, and a temperature change member disposed with the absorbent body. The temperature change member includes a temperature change member first surface and a temperature change member second surface opposite the temperature change member first surface. The temperature change member also includes a temperature change material disposed between the first surface and the second surface-where the temperature change first surface is in an overlaid relationship with itself to define a first folded region, a second folded region, and an unfolded region disposed between the first folded region and the second folded region. The temperature change member also provides the article with a temperature change of at least 5 degrees C., as determined by the temperature change test described herein.

In yet another aspect, the present invention is directed to an absorbent article defining an article first side edge and an article second side edge opposite the article first side edge and a pair of end edges. The absorbent article includes a liquid impermeable outercover, an absorbent body disposed on the outercover, and a temperature change member disposed with the absorbent body.

The temperature change member includes a temperature change material and a plurality of layers in at least a portion of the temperature change member where the temperature change member provides the article with a temperature change of at least 5 degrees C., as determined by the temperature change test described herein.

The above-mentioned and other aspects of the present invention will become more apparent, and the invention itself will be better understood by reference to the drawings and the following description of the drawings.

### BRIEF DESCRIPTION OF THE DRAWINGS

FIG. 1 representatively illustrates a side view of a pair of training pants with a mechanical fastening system of the pants shown fastened on one side of the training pants and unfastened on the other side of the training pants;

FIG. 2 representatively illustrates a plan view of the training pants of FIG. 1 in an unfastened, stretched and laid flat condition, and showing the surface of the training pants that faces away from the wearer;

FIG. 3 representatively illustrates a plan view similar to FIG. 2, but showing the surface of the training pants that faces the wearer when worn, and with portions cut away to show underlying features;

FIG. 4 representatively illustrates a section view of a particular. aspect of the temperature change member of the present invention with the folded regions omitted for clarity;

FIG. 5 representatively illustrates a section view of another aspect of the temperature change member of the present invention with the folded regions omitted for clarity;

FIG. 6 representatively illustrates a section view of a particular aspect of the training pants with certain optional elements removed for clarity;

FIG. 7 representatively illustrates a section view of another aspect of the training pants with certain optional elements removed for clarity; and

FIG. 8 representatively illustrates a section view of yet another aspect of the training pants with certain optional elements removed for clarity.

Corresponding reference characters indicate corresponding parts throughout the drawings.

Definitions

Within the context of this specification, each term or phrase below includes the following meaning or meanings:

“Attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Coform” refers to a blend of meltblown fibers and absorbent fibers such as cellulosic fibers that can be formed by air forming a meltblown polymer material while simultaneously blowing air-suspended fibers into the stream of meltblown fibers. The coform material may also include other materials, such as superabsorbent materials. The meltblown fibers and absorbent fibers are collected on a forming surface, such as provided by a foraminous belt. The forming surface may include a gas-pervious material that has been placed onto the forming surface.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Elastic,” “elasticized,” “elasticity,” and “elastomeric” mean that property of a material or composite by virtue of which it tends to recover its original size and shape after removal of a force causing a deformation. Suitably, an elastic material or composite can be elongated by at least 50 percent (to 150 percent) of its relaxed length and will recover, upon release of the applied force, at least 40 percent of its elongation.

“Extensible” refers to a material or composite which is capable of extension or deformation without breaking, but does not substantially recover its original size and shape after removal of a force causing the extension or deformation. Suitably, an extensible material or composite can be elongated by at least 50 percent (to 150 percent) of its relaxed length.

“Fiber” refers to a continuous or discontinuous member having a high ratio of length to diameter or width. Thus, a fiber may be a filament, a thread, a strand, a yarn, or any other member or combination of these members.

“Hydrophilic” describes fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 degrees are designated “wettable” or hydrophilic, and fibers having contact angles greater than 90 degrees are designated “nonwettable” or hydrophobic.

“Join” and its derivatives refer to the connecting, adhering, bonding, attaching, sewing together, or the like, of two elements. Two elements will be considered to be joined together when they are integral with one another or joined directly to one another or indirectly to one another, such as when each is directly joined to intermediate elements. “Join” and its derivatives include permanent, releasable, or refastenable joinder. In addition, the joining can be completed either during the manufacturing process or by the end user.

“Layer” when used in the singular can have the dual meaning of a single element or a plurality of elements.

“Liquid impermeable,” when used in describing a layer or multi-layer laminate means that liquid, such as urine, will not pass through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Such a process is disclosed, for example, in U.S. Pat. No. 3,849,241 to Butin et al. Meltblown fibers may be continuous or discontinuous and are generally self bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

“Stretchable” means that a material can be stretched, without breaking, by at least 50 percent (to 150 percent of its initial (unstretched) length) in at least one direction. Elastic materials and extensible materials are each stretchable materials.

“superabsorbent material” refers to a water-swellable, water-insoluble organic or inorganic material capable, under the most favorable conditions, of absorbing at least about ten times its weight and, more desirably, at least about thirty times its weight in an aqueous solution containing about 0.9 weight percent sodium chloride.

These terms may be defined with additional language in the remaining portions of the specification.

### DETAILED DESCRIPTION

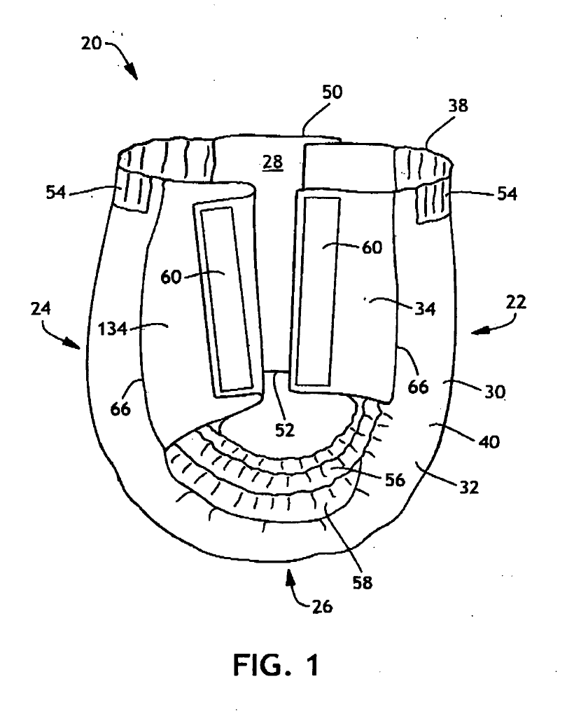

Referring now to the drawings and in particular to FIG. 1, an absorbent article of the present invention is representatively illustrated in the form of children's toilet training pants and is indicated in its entirety by the reference numeral 20. The pants 20 include a temperature change member 70 that is adapted to create a distinct temperature change sensation to the wearer upon urination, which can enhance a wearer's ability to recognize when urination is occurring. In addition, the temperature change member 70 defines a first folded region 82 and optionally a second folded region 84 to further improve the performance of the temperature change member 70 by enhancing its ability to maintain close body contact with the wearer in use.

The pants 20 may or may not be disposable, which refers to articles that are intended to be discarded after a limited period of use instead of being laundered or otherwise conditioned for reuse. It should also be understood that the present invention can be suitable for use with various other absorbent articles intended for personal wear, including but not limited to diapers, feminine hygiene products, incontinence products, medical garments, surgical pads and bandages, other personal care or health care garments, and the like without departing from the scope of the present invention.

By way of illustration only, various materials and methods for constructing training pants such as the pants 20 of the various aspects of the present invention are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference to the extent that they are consistent (i.e., not in conflict) herewith. In addition, absorbent articles including a temperature change member are described in U.S. Pat. No. 5,681,298 to Brunner et al., and U.S. patent application Ser. No. 11/143359 filed Jun. 1, 2005 in the name of Jackson, et al., the disclosures of which are incorporated herein by reference to the extent that they are consistent (i.e., not in conflict) herewith.

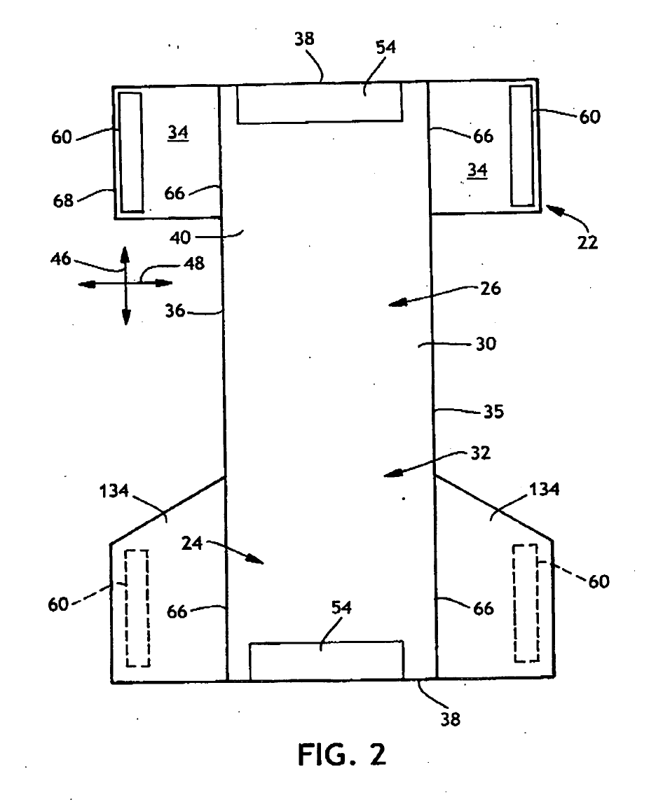

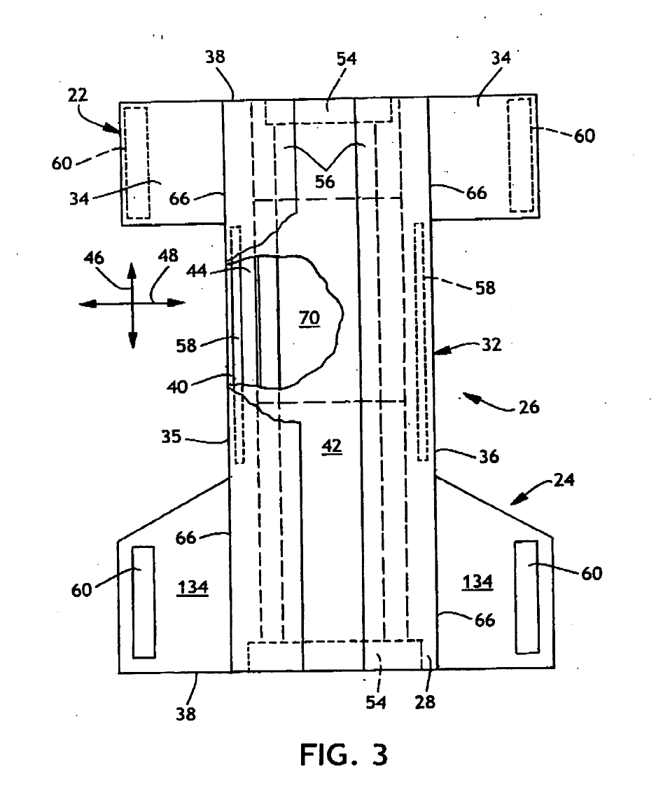

The training pants 20 are illustrated in FIG. 1 in a partially fastened condition. The pants 20 define a longitudinal direction 46 and a lateral direction 48 perpendicular to the longitudinal direction as shown in FIGS. 2 and 3. The pants 20 further define a pair of longitudinal end regions, otherwise referred to herein as a front waist region 22 and a back waist region 24, and a center region, otherwise referred to herein as a crotch region 26, extending longitudinally between and interconnecting the front and back waist regions 22, 24. The front and back waist regions 22, 24 includes those portions of the pants 20, which when worn, wholly or partially cover or encircle the waist or mid-lower torso of the wearer. The crotch region 26 generally is that portion of the pants 20 which, when worn, is positioned between the legs of the wearer and covers the lower torso and crotch of the wearer. The pants 20 also define an inner surface 28 adapted in use to be disposed toward the wearer, and an outer surface 30 opposite the inner surface. With additional reference to FIGS. 2 and 3, the pair of training pants 20 has a laterally extending first side edge 35 and a laterally extending second side edge 36 opposite the first side edge, and a pair of longitudinally opposite waist edges 38 (broadly, end edges).

The illustrated pants 20 can include an absorbent assembly, generally indicated at 32. For example, in the aspect of FIGS. 1-3, the training pants 20 include a generally rectangular central absorbent assembly 32 and side panels 34, 134 formed separately from and secured to the central absorbent assembly. The side panels 34, 134 can be bonded along seams 66 to the absorbent assembly 32 in the respective front and back waist regions 22 and 24 of the pants 20. More particularly, the front side panels 34 can be permanently bonded to and extend laterally outward from the absorbent assembly 32 at the front waist region 22, and the back side panels 134 can be permanently bonded to and extend laterally from the absorbent assembly 32 at the back waist region 24. The side panels 34 and 134 may be bonded to the absorbent assembly 32 using attachment means known to those skilled in the art such as adhesive, thermal or ultrasonic bonding.

The front and back side panels 34 and 134, upon wearing of the pants 20, thus include the portions of the training pants 20 that are positioned on the hips of the wearer. The front and back side panels 34 and 134 can be permanently bonded together to form the three-dimensional configuration of the pants 20, or be releasably connected with one another such as by a fastening system 60 of the illustrated aspects.

Suitable elastic materials, as well as processes for incorporating elastic side panels into training pants, are described in the following U.S. Pat. No.: 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference in their entirety to the extent they are consistent (i.e., not in conflict) herewith. In particular aspects, the elastic material may include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference in their entirety to the extent they are consistent (i.e., not in conflict) herewith. As is known in the art, the side panels 34, 134 may include elastic material or stretchable but inelastic materials.

The absorbent assembly 32 is illustrated in FIGS. 2-3 as having a rectangular shape. However, it is contemplated that the absorbent assembly 32 may have other shapes (e.g., hourglass, T-shaped, I-shaped, and the like) without departing from the scope of this invention. It is also understood that the side panels 34, 134 may alternatively be formed integrally with the absorbent assembly 32 without departing from the scope of this invention. In such a configuration, the side panels 34 and 134 and the absorbent assembly would include at least some common materials, such as the bodyside liner 42, outercover 40, other materials and/or combinations thereof.

The absorbent assembly 32 includes an outercover 40 and a bodyside liner 42 (FIG. 3, 6-8) in a superposed relation therewith. The liner 42 can be suitably joined to the outercover 40 along at least a portion of the longitudinal ends of the pants 20. The liner 42 can be suitably adapted, i.e., positioned relative to the other components of the pants 20, to contact the wearer's skin during wear of the pants. The absorbent assembly 32 also includes an absorbent body 44 (FIG. 3, 6-8) disposed between the outercover 40 and the bodyside liner 42 for absorbing liquid body exudates. The liner 42 can be suitably joined to the outercover 40 along at least a portion of the absorbent assembly 32. The bodyside liner 42 and the outercover 40 can, for example, be attached to each other by adhesive, ultrasonic bonding, thermal bonding or by other suitable attachment techniques known in the art. Moreover, at least a portion of the absorbent body 44 can optionally be attached to the bodyside liner 42 and/or the outercover 40 utilizing the methods described above.

As mentioned above, the front and back side panels 34 and 134 can be releasably connected with one another such as by the fastening system 60 of the illustrated aspect. With the training pants 20 in the fastened position as partially illustrated in FIG. 1, the front and back waist regions are connected together to define the three-dimensional pants configuration having a waist opening 50 and a pair of leg openings 52. The waist edges 38 (e.g., longitudinal ends) of the training pants 20 are configured to encircle the waist of the wearer to define the waist opening 50 (FIG. 1) of the pants.

The fastening system 60 may include any refastenable fasteners suitable for absorbent articles, such as adhesive fasteners, cohesive fasteners, mechanical fasteners, or the like. In one aspect of the invention, the fastening system includes mechanical fastening elements for improved performance. Suitable mechanical fastening elements can be provided by interlocking geometric-shaped materials, such as hooks, loops, bulbs, mushrooms, arrowheads, balls on stems, male and female mating components, buckles, snaps, or the like. For example, fastening systems are also disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

The pants 20 may further include a pair of containment flaps 56 for inhibiting the lateral flow of body exudates. As illustrated in FIG. 3, the containment flaps 56 can be operatively attached to the pants 20 in any suitable manner as is well known in the art. In particular, suitable constructions and arrangements for the containment flaps 56 are generally well known to those skilled in the art and are described in U.S. Pat. No. 4,704,116 issued Nov. 3, 1987 to Enloe, which is incorporated herein by reference to the extent that it is consistent (i.e., not in conflict) herewith.

To further enhance containment and/or absorption of body exudates, the training pants 20 may include waist elastic members 54 in the front and/or back waist regions 22 and 24 of the pants 20. Likewise, the pants 20 may include leg elastic members 58, as are known to those skilled in the art. The waist elastic members 54 and the leg elastic members 58 can be formed of any suitable elastic material that is well known to those skilled in the art. For example, suitable elastic materials include sheets, strands or ribbons of natural rubber, synthetic rubber, or thermoplastic elastomeric polymers. In one aspect of the invention, the waist elastics and/or the leg elastics may include a plurality of dry-spun coalesced multi-filament elastomeric threads sold under the trade name LYCRA and available from Invista of Wilmington, Del., U.S.A.

The outercover 40 may suitably include a material that is substantially liquid impermeable. The outercover 40 may be provided by a single layer of liquid impermeable material, or more suitably include a multi-layered laminate structure in which at least one of the layers is liquid impermeable. In particular aspects, the outer layer may suitably provide a relatively cloth-like texture to the wearer. A suitable liquid impermeable film for use as a liquid impermeable inner layer, or a single layer liquid impermeable outercover 40 is a 0.025 millimeter (1.0 mil) polyethylene film commercially available from Edison Plastics Company of South Plainfield, N.J. Alternatively, the outercover 40 may include a woven or non-woven fibrous web layer that has been totally or partially constructed or treated to impart the desired levels of liquid impermeability to selected regions that are adjacent or proximate the absorbent body.

The outercover 40 may also be stretchable, and in some aspects it may be elastomeric. For example, such an outercover material can include a 0.3 osy polypropylene spunbond that is necked 60 percent in the lateral direction 40 and creped 60 percent in the longitudinal direction 48, laminated with 3 grams per square meter (gsm) Bostik-Findley H2525A styrene-isoprene-styrene based adhesive to 8 gsm PEBAX 2533 film with 20 percent TiO₂ concentrate. Reference is made to U.S. Pat. No. 5,883,028, issued to Morman et al., U.S. Pat. No. 5,116,662 issued to Morman and U.S. Pat. No. 5,114,781 issued to Morman, all of which are hereby incorporated herein by reference, for additional information regarding suitable outercover materials.

The bodyside liner 42 is suitably compliant, soft-feeling, and non-irritating to the wearer's skin. The bodyside liner 42 is also sufficiently liquid permeable to permit liquid body exudates to readily penetrate through its thickness to the absorbent body 44. A suitable liquid permeable bodyside liner 42 is a nonwoven polyethylene/polypropylene bicomponent web having a basis weight of about 27 gsm; the web may be spunbonded or a bonded carded web. Optionally, the bodyside liner 42 may be treated with a surfactant to increase the wettability of the liner material.

Alternatively, the bodyside liner 42 may also be stretchable, and in some aspects it may be elastomeric. For instance, the liner 42 can be a non-woven, spunbond polypropylene fabric composed of about 2 to 3 denier fibers formed into a web having a basis weight of about 12 gsm which is necked approximately 60 percent. Strands of about 9 gsm KRATON G2760 elastomer material placed eight strands per inch (2.54 cm) can be adhered to the necked spunbond material to impart elasticity to the spunbond fabric. The fabric can be surface treated with an operative amourit of surfactant, such as about 0.6 percent AHCOVEL Base N62 surfactant, available from ICI Americas, a business having offices in Wilmington, Del., U.S.A. Other suitable materials may be extensible biaxially stretchable materials, such as a neck stretched/creped spunbond. Reference is made to U.S. Pat. No. 6,552,245, issued Apr. 22, 2003, to Roessler et al., which is incorporated by reference herein to the extent that it is consistent (i.e., not in conflict) herewith.

An absorbent body 44 may be disposed on the outercover 40, for example, between the outercover 40 and the bodyside liner 42. The outercover 40 and the bodyside liner 42 can be joined together by any suitable means such as adhesives, ultrasonic bonds, thermal bonds, or the like. The absorbent body 44 can be in a variety of shapes and configurations as are known in the art, such as rectangular, hourglass shaped, I-shaped, and the like. Further, at least a portion of the absorbent body 44 can optionally be attached to the bodyside liner 42 and/or the outercover 40 utilizing the methods described above.

The absorbent body 44 is suitably compressible, conformable and capable of absorbing and retaining liquid body exudates released by the wearer. For example, the absorbent assembly can include a matrix of absorbent fibers, and more suitably cellulosic fluff, such as wood pulp fluff, and superabsorbent particles. One suitable pulp fluff is identified with the trade designation CR1654, commercially available from Bowater, Inc. of Greenville, S.C., U.S.A. As an alternative to wood pulp fluff, synthetic fibers, polymeric fibers, meltblown fibers, short cut homofil bicomponent synthetic fibers, or other natural fibers may be used. Suitable superabsorbent materials can be selected from natural, synthetic, and modified natural polymers and materials. The superabsorbent materials can be inorganic materials, such as silica gels, or organic compounds, such as crosslinked polymers, for example, sodium neutralized polyacrylic acid. Suitable superabsorbent materials are available from various commercial vendors, such as Dow Chemical Company of Midland, Mich. U.S.A., and Stockhausen Inc., Greensboro, N.C. U.S.A.

In one aspect, the absorbent body 44 may be stretchable so as not to inhibit the stretchability of other components to which the absorbent body may be adhered, such as the outercover 40 and/or the bodyside liner 42. For example, the absorbent body may include materials disclosed in U.S. Pat. Nos. 5,964,743, 5,645,542, 6,231,557, 6,362,389, and international patent application WO 03/051254, the disclosure of each of which is incorporated by reference herein.

In some aspects, a surge management layer (not shown) may be included in the pants 20. The surge management layer may be positioned in the pants 20 in a variety of locations as is known in the art. For example, the surge management layer can be proximate the absorbent body 44, for example between the absorbent body 44 and the bodyside liner 42, and attached to one or more components of the pants 20 by methods known in the art, such as by adhesive, ultrasonic or thermal bonding. In addition, the surge management layer may be positioned in the pants 20 relative to the temperature change member 70 in a variety of ways. For instance, the surge management layer may be disposed toward the liner 42 relative to the temperature change member 70, or the surge management layer may be disposed toward the absorbent body 44 relative to the temperature change member 70.

A surge management layer helps to decelerate and diffuse surges or gushes of liquid that may be rapidly introduced into the absorbent body 44. Desirably, the surge management layer can rapidly accept and temporarily hold the liquid prior to releasing the liquid into the storage or retention portions of the absorbent body 44. Examples of suitable surge management layers are described in U.S. Pat. No. 5,486,166 and U.S. Pat. No. 5,490,846, the contents of which are incorporated herein by reference to the extent that they are consistent (i.e., not in conflict) herewith.

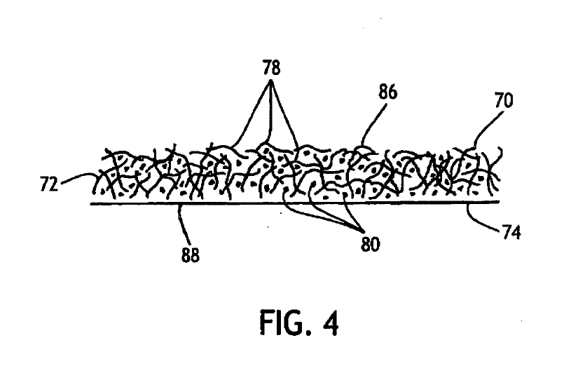

As mentioned above, the various aspects of the absorbent article of the present invention can also include a temperature change member 70 (FIG. 3, 6-8). The temperature change member 70 can include a temperature change member first surface and a temperature change member second surface opposite the first surface. For example, as representatively illustrated in FIGS. 4-8, the temperature change member 70 can include a temperature change member bodyfacing surface 86 and a temperature change member garment facing surface 88 opposite the bodyfacing surface 86.

Moreover, the temperature change member 70 can define a folded region such as a first folded region 82. Optionally, the temperature change member can define a plurality of folded regions. For example, as representatively illustrated in FIGS. 3, 6-8, the temperature change member 70 can define the first folded region 82 and a second folded region 84. Alternatively, it is contemplated that the temperature change member can include three or more folded regions and still remain within the scope of the present invention.

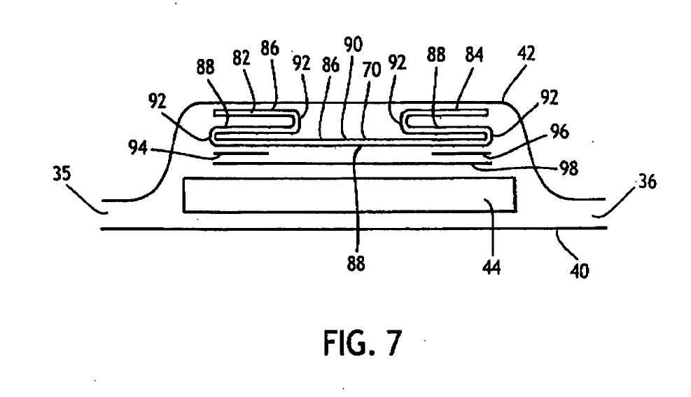

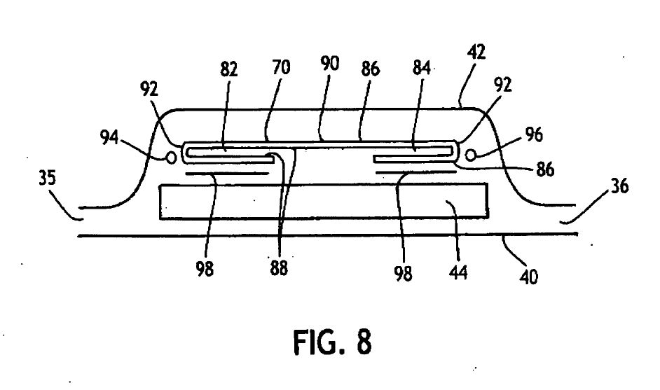

The folded regions 82 and 84 can be provided in a variety of ways as can be appreciated by one of ordinary skill in the art. For example, one of the temperature change member surfaces 86, 88 can be provided in an overlaid relationship with itself. Specifically, in one aspect, the temperature change member bodyfacing surface 86 can be in an overlaid relationship with itself to define the first folded region 82 and/or the second folded region 84 (FIG. 3, 6, 7). Alternatively, in another aspect, the temperature change member garment facing surface 88 can be in an overlaid relationship with itself to define the first folded region and/or the second folded region (FIGS. 7 and 8). In yet another aspect, both the temperature change member garment facing surface 88 can be in an overlaid relationship with itself to provide one folded region 82 or 84 and the temperature change member bodyfacing surface can also be in an overlaid relationship with itself to provide another folded region 82 or 84. Alternatively, and as will be described in greater detail below, one or more folded regions 82 or 84 can include a plurality of folds (FIG. 7). Thus, as described above with regard to the folded regions 82 and 84, the temperature change member 70 can include a plurality of layers in at least a portion of the temperature change member 70.

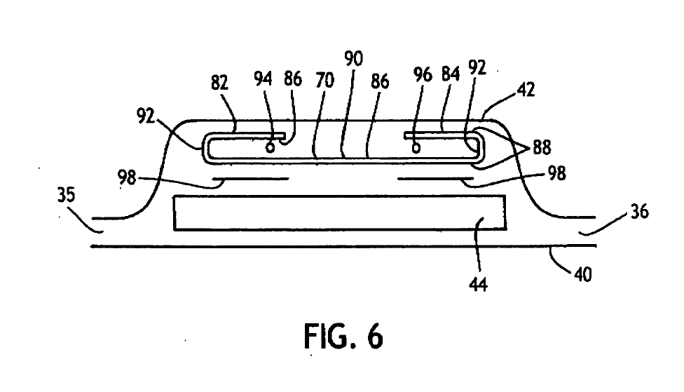

As discussed above, the surfaces 86 and 88 of the temperature change member can be overlaid upon themselves to define the temperature change member folded regions 82 and 84 of the present invention. As can be readily appreciated, the surfaces 86 and 88 can be in a direct facing relationship to provide the folded regions (FIGS. 6-8). Nonetheless, it should be readily appreciated that other layers or other materials can be intermediate the surfaces 86 and 88 within the folded regions 82 and 84 and still be within the scope of the present invention.

The folded regions 82 and 84 of the present invention can be provided in a number of configurations as are known in the art. For example, the folded regions 82, 84 can be provided in a C-fold configuration for improved performance and ease of manufacture (FIGS. 6, 8). Alternatively, the folded regions 82 and 84 can be provided in a Z-fold configuration for increased rigidity (FIG. 7). As can be readily appreciated, in configurations where the temperature change member 70 defines a plurality of folded regions 82, 84, the folded regions 82, 84 can be provided in the same folding arrangement or in different folding arrangements. For example, the first folded region 82 and the second folded region 84 can each be provided in a C-fold configuration (FIG. 6, 8) or a Z-fold configuration (FIG. 7). Alternatively, the first folded region 82 can be provided in a C-fold configuration while the second folded region 84 can be provided in a different fold configuration such as a Z-fold configuration.

Due to the increased sturdiness in the folded regions 82 and 84 resulting from the multiple layers of temperature change member 70 in those regions, the folded regions 82 and 84 advantageously are capable of resisting deformation in use. As such, at least the folded regions 82 and 84 of the temperature change member 70 have a greater likelihood of remaining in close body contact with the wearer when the pants 20 are squeezed between the legs of the wearer. That is, when the wearer squeezes the pants 20 between their legs, the temperature change member 70 may have a tendency to buckle with the rest of the crotch region 26 of the pants 20 and as a result, be forced away from the skin of the wearer. Nonetheless, with the temperature change member 70 of the present invention, the folded regions 82 and 84 are more likely to resist buckling and remain in place (i.e., closer to the body of the wearer) and therefore be more effective in providing a temperature change to the wearer upon urination. Moreover, the cooling effect of the temperature change member 70 can be concentrated in the folded regions due to the multiple layers of the member 70 that are present in the folded regions 82, 84. In particular, the folded regions may be better adapted to provide a cooling sensation in response to repeated urination episodes and provide a greater change in temperature experienced by the wearer due to the concentration of the temperature change member 70 in the folded regions 82, 84.

As mentioned above, the temperature change member can define at least a first folded region 82, and can optionally further define a second folded region 84. Further, as representatively illustrated in FIGS. 3, 6-8, the temperature change member can optionally define an unfolded region 90. For example, a portion of the temperature change member 70 can be in a folded arrangement to define a folded region while another portion of the temperature change member 70 can be in a substantially planar condition to define an unfolded region 90. In a particular aspect where the temperature change member defines a plurality of folded regions, such as the first folded region 82 and the second folded region 84, the unfolded region 90 can be disposed between the first folded region and the second folded region (FIGS. 3, 6-8).

The folded regions 82 and 84 can include a fold axis 92 about which the temperature change member surfaces 86 and 88 can be placed in an overlaid relationship with themselves. As representatively illustrated in FIG. 3, the fold axis 92 can be substantially linear and extend substantially in the longitudinal direction 46. Alternatively, the fold axis 92 can be curvilinear, which may be advantageous to provide a tailored shape to the temperature change member 70, such as proximate the leg openings 52. In another alternative, the fold axis 92 may extend in substantially the lateral direction 48, or optionally can extend at an angle from the longitudinal and/or lateral directions 46 and 48.

The folded regions 82 and 84 can be located in a variety of locations in the temperature change member 70 such that they can advantageously enhance the tactile feedback to the wearer in use. For example, as representatively illustrated in FIGS. 3, 6-8, the first folded region 82 can be optionally located proximate the article first side edge 35. Similarly, in aspects where the temperature change member includes a plurality of folded regions 82, 84, the second folded region 84 can optionally be proximate the article second side edge 36. As such, in use, the folded regions 82 and 84 can be located in crotch region 26 of the pants 20 that are less likely to buckle and deform in use and thus remain closer to the body of the wearer.

The temperature change member 70 of the pants 20 of the present invention can also define a temperature change member area in its folded condition. That is, the temperature change member area is the surface area of the temperature change member 70 in its folded condition. Likewise, the folded regions 82 and 84 can also define folded region areas, which is the surface area of the folded regions 82 and 84 in their folded state. For example, the first folded region 82 can define a first folded region area, and in aspects where the temperature change member 70 includes a plurality of folded regions, the second folded region 84 can define a second folded region area. Moreover, the individual folded region areas can be summed to define a total folded region area. For example in aspects where the temperature change member 70 includes a first folded region 82 and a second folded region 84, the total folded region area would be the sum of the first folded region area and the second folded region area.

Accordingly, the first folded region 82 can define a first folded region area that is at least 25 percent of the temperature change member area, and alternatively at least 33 percent of the temperature change member area for improved temperature change performance in use. Likewise, in aspects where the temperature change member 70 includes a second folded region 84, the second folded region 84 can define a second folded region area that is at least 25 percent of the temperature change member area, and alternatively at least 33 percent of the temperature change member area for improved temperature change performance in use. Further, the total folded region area can be at least 33percent and alternatively at least 50 percent of the temperature change member area for improved temperature change performance in use.

The various areas described above can be determined using methods known in the art. For example, where the area to be measured is generally rectilinear in shape, the area may be determined by multiplying the largest dimension of the area in the lateral direction 48 by the largest dimension of the area in the longitudinal direction 46. Alternatively, for substantially non-rectilinear areas, the area of respective regions can be measured by positioning a sufficiently large piece of paper of known and even basis weight on the article, and then marking the limits of the regions. Subsequent cutting and accurate weighing provides the weight of the paper sheet, which by dividing through the basis weight results in the area of the paper and therefore of the respective region.

In another aspect of the present invention, the pants 20 can advantageously include an elastic member disposed with the temperature change member 70 to further improve the performance of the member 70 in use. In a particular aspect, a first elastic member 94 can be disposed with the first folded region 82 (FIGS. 6-8). As such, the elastic member 94 can assist in maintaining at least a portion of the temperature change member in close body contact with the wearer in use. In a particular aspect where the temperature change member 70 includes a plurality of folded regions such as the first and second folded regions 82 and 84, the first elastic member 94 and a second elastic member 96 can be disposed with each of the respective folded regions 82 and 84.

Elastic members 94 and 96 can be disposed with the folded regions 82 and 84 in a number of ways as are known in the art. For example, the elastic members 94 and 96 can be placed adjacent the folded regions (FIG. 8), beneath the folded regions (FIG. 7) or the elastic members can be sandwiched within the folded regions 82 and 84 (FIG. 6) or combinations thereof. Further, in aspects where the temperature change member 70 includes a plurality of folded regions such as the first and second folded regions 82 and 84, the elastic members 94 and 96 may be disposed with the folded regions 82 and 84 in the same manner, or alternatively, the elastic members 94 and 96 can be disposed with the folded regions 82 and 84 in differing manners.

The elastic members 94 and 96 can be provided by a variety of elastic materials as are known in the art. For example, strands of elastic fiber such as LYCRA available from Invista, a company having offices in Wichita, KS, can be used for the elastic members 94 and 96. Alternatively, the elastic members 94 and 96 can be provided by strands, ribbons or sheets of elastic materials such as NBL, STL, or SBL (described above in connection with the side panels 34, 134) or combinations thereof. In still yet another alternative, the elastic members 94 and 96 can be provided by a combination of the above mentioned or other suitable elastic materials.

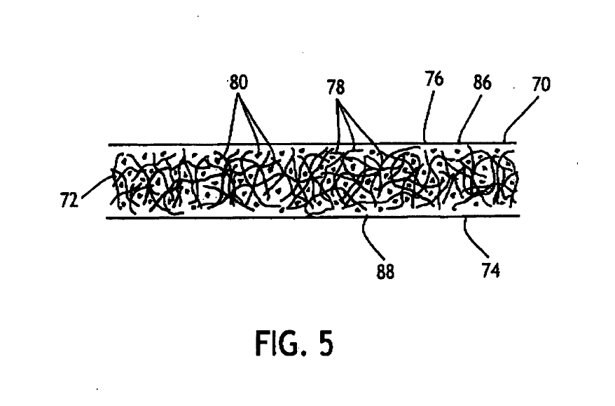

The temperature change member 70 can include a temperature change composite 72 and optionally a first carrier layer 74 in a superposed relationship with temperature change composite 72 (FIGS. 4 and 5). The temperature change member 70 can also optionally include a second carrier layer 76 where the first carrier layer 74 and the second carrier layer 76 sandwich the temperature change composite 72 (FIG. 5). As such, in aspects where the temperature change member 70 does not include carrier layers 74 or 76, the composite 72 can provide the surfaces 86 and 88 of the temperature change member 70. Alternatively, when present, the carrier layers 74 and 76 can provide one or both of the temperature change member surfaces 86 and 88.

The temperature change composite 72 includes temperature change material 80 and optionally, a matrix of fibers 78 where the temperature change material 80 is intermixed within the matrix of fibers 78. The matrix of fibers 78 may be substantially continuous or discrete and discontinuous. In addition, the matrix of fibers 78 of the temperature change composite 72 may be provided by a variety of different fibers as are known in the art. For example, the matrix of fibers 78 can include adhesive fibers, absorbent fibers, binders (including binder fibers), polymer fibers, and the like or combinations thereof. As such, the temperature change material 80 may be suitably entrapped within the matrix 78 to limit material shake-out or loss during manufacture and/or wear of the pants 20. Suitable temperature change composites 72 are described in U.S. Pat. No. 5,681,298 and U.S. Pat. application Ser. No. 11/143359, each of which has been previously incorporated herein.

In particular, in aspects where the matrix of fibers 78 includes adhesive fibers, the fibers may be provided by a hot-melt adhesive. Such an adhesive generally comprises one or more polymers to provide cohesive strength, a resin or analogous material, perhaps waxes, plasticizers or other materials to modify viscosity, and/or other additives including, but not limited to, antioxidants or other stabilizers. It is also contemplated that alternative adhesives may be used without departing from the scope of this invention.

The temperature change material 80 can be intermixed with the adhesive fibers providing the matrix of fibers 78 by being fed into and entrained in a stream of adhesive to form a blended mixture of adhesive fibers and temperature change material 80. In such an aspect, the matrix of fibers can be optionally applied to a substrate, such as the first carrier layer 74. In addition, the second carrier layer 76 can, but need not, overlay the temperature change composite 72 and be secured thereto by the adhesive in the matrix of fibers 78.

An example of a suitable adhesive for use in providing the matrix of fibers 78 are hot-melt adhesives available from H. B. Fuller Adhesives of Saint Paul, Minn. under the designation HL8151-XZP. In particular, this adhesive is a hydrophilic adhesive which promotes the rapid wettability of the temperature change member 70 resulting in faster temperature change. Alternatively, it is contemplated that the adhesive can be a hydrophobic adhesive without departing from the scope of the present invention.

Alternatively, the matrix of fibers 78 of the temperature change composite 72 can include absorbent fibers. In such an aspect, the matrix of fibers 78 may be provided by absorbent fibers by forming the matrix on a forming surface of a conventional air-forming device. Suitable absorbent fibers can include natural absorbent fibers such as cellulosic fibers (i.e., wood pulp fibers) or cotton fibers, synthetic absorbent fibers such as rayon or cellulose acetate or combinations thereof. In particular, the absorbent fibers can be a mixed bleached southern softwood and hardwood Kraft pulp designated as CR1654 available from Bowater Inc. of Greenville, S.C. U.S.A. Other suitable absorbent fibers can include NB 416, a bleached southern softwood Kraft pulp available from Weyerhaeuser Co. of Federal Way, Wash. U.S.A.; CR 1654, a bleached southern softwood Kraft pulp available from Bowater, Incorporated, of Greenville, S.C. U.S.A.; SULPHATATE HJ, a chemically modified hardwood pulp available from Rayonier Inc. of Jesup, Ga. U.S.A. and NF 405, a chemically treated bleached southern softwood Kraft pulp available from Weyerhaeuser Co.

Optionally, in such an aspect, the matrix of fibers 78 can further include binder material. For example, the binder material can suitably be a thermoplastic binder material. Such binder materials can soften when exposed to heat and can substantially returns to their original condition when cooled to room temperature. Such thermoplastic binder materials, when in the softened state, constrain or entrap the fibers and other materials proximate the binder to stabilize the temperature change composite 72. Binder materials can be provided in powder or fiber form. Examples of suitable binder materials for use with the present invention can be those having low melting temperatures such as polyethylene glycol (PEG) or paraffin wax, both of which are available from Alrich of Saint Louis, MO.

In yet another alternative the matrix of fibers 78 may be provided by a coform composite including polymer fibers and absorbent fibers. Coform materials and coforming processes are known in the art and by way of example are described in U.S. Pat. No. 4,100,324 to Anderson, et al.; U.S. Pat. No. 5,284,703 Everhart, et al.; and U.S. Pat. No. 5,350,624 to Georger, et al.; each of which are hereby incorporated by reference to the extent that they are consistent (i.e., not in conflict) herewith.

In a particular aspect, the matrix of fibers 78 may be provided by a coform composite that can be a blend of meltblown polymer fibers and cellulosic fibers. Various suitable materials may be used to provide the meltblown fibers such as a polyolefin material. Alternatively, the polymer fibers may be stretch polymer fibers, such as those provided by a copolymer resin. For instance, Vistamaxx® elastic olefin copolymer resin designated PLTD-1819 available from ExxonMobil Corporation of Houston, Tex. or KRATON G-2755 available from Kraton Polymers of Houston, Tex. may be used to provide stretchable polymer fibers for the matrix of fibers 78. Other suitable polymeric materials or combinations thereof may alternatively be utilized as are known in the art.

Further, various absorbent cellulose fibers may be utilized, such as NF 405, a chemically treated bleached southern softwood Kraft pulp available from Weyerhaeuser Co. of Federal Way, Wash. U.S.A.; NB 416, a bleached southern softwood Kraft pulp available from Weyerhaeuser Co.; CR-0056, a fully debonded softwood pulp available from Bowater Inc. of Greenville S.C., Golden Isles 4822 debonded softwood pulp available from Koch Cellulose of Brunswick, Ga., U.S.A.; and SULPHATATE HJ, a chemically modified hardwood pulp available from Rayonier Inc. of Jesup, Ga. U.S.A.

The polymer fibers and the meltblown fibers may be coformed to provide the matrix of fibers 78 by providing a stream of absorbent fibers and a stream of extruded molten polymeric fibers. Further, to provide the temperature change composite 72, a stream of temperature change material 80 can also be provided. These streams can be merged into a single stream and collected on a forming surface such as a forming belt or forming drum to form the temperature change composite 72 of the temperature change member 70. Optionally, a forming layer, such as first carrier layer 74, can be placed on the forming surface and used to collect the materials included in the temperature change composite 72.

The stream of absorbent fibers may be provided by feeding a pulp sheet into a fiberizer, hammermill, or similar device as are known in the art. Suitable fiberizers are available from Hollingsworth of Greenville, S.C. and are described in U.S. Pat. No. 4,375,448 issued Mar. 1, 1983 to Appel et al. The stream of polymer fibers may be provided by meltblowing a copolymer resin or other polymer. In particular, the melt temperature for a copolymer resin such as Vistamaxx® PLTD 1810 can be from 450 degrees F. (232 degrees C.) to 540 degrees F. (282 degrees C.) to improve the entrainment of the temperature change material in the matrix. As mentioned above, suitable techniques for producing nonwoven fibrous webs, which include meltblown fibers, are described in the previously incorporated U.S. Pat. Nos. 4,100,324 and 5,350,624. The meltblowing techniques can be readily adjusted in accordance with conventional know-how to provide turbulent flows that can operatively intermix the fibers and the temperature change material 80. For example, the primary air pressure may be set at 5 psi and the meltblown nozzles may be 0.020 inch spinneret hole nozzles. The techniques can also be readily adjusted in accordance with conventional knowledge to provide the desired weight percentages of the various materials in the temperature change composite 72.

The stream of temperature change material 80 may be pneumatically provided or gravity fed. A suitable method and apparatus for delivering material in an airstream is described in U.S. Pat. No. 4,604,313 issued Aug. 5, 1986 to McFarland et al.; the disclosure of which is incorporated by reference herein to the extent that it is consistent (i.e., not in conflict) herewith. The coform material may also include other materials, such as superabsorbent materials.

In one aspect, the temperature change composite 72 provided by a coform composite as described above may be from 5 to 15 percent by weight meltblown polymer fibers, 10 to 50 percent by weight absorbent fibers and 40 to 80 percent by weight temperature change material. In a particular aspect, the temperature change composite 72 can be 8 percent by weight meltblown polymer fibers, 14 percent by weight absorbent fibers, 78 percent by weight temperature change material and define a basis weight of 1340 gsm.

In particular aspects where the temperature change composite 72 is provided by a coform composite as described above, portions of the temperature change composite can act as a distribution layer similar to the surge management layer described above. That is, upon one or more insults, the temperature change material 80 that was present in the insult area can be consumed, but the remaining coform material in the temperature change composite can act to distribute future insults both to other regions of the temperature change member 70, thereby more effectively alerting the wearer to future insults, and to other regions of the absorbent body 44 to improve the performance of the pants 20.

As mentioned above, the temperature change member 70 can optionally include a first carrier layer 74 (FIGS. 4 and 5) in superposed relationship with the temperature change composite 72. Further, the temperature change member 70 can optionally include a first carrier layer 74 and a second carrier layer 76 (FIG. 5) where the first carrier layer and the second carrier layer 76 sandwich the temperature change composite 72. The first and second carrier layers 74 and 76 may be provided by separate webs of material, or alternatively can be provided by a single web of material that is folded in half about the temperature change composite 72.

In certain aspects, the carrier layers 74 and 76 can be liquid permeable or liquid impermeable. For instance, one carrier layer, such as the first carrier layer 74 may be at least partially liquid impermeable, and optionally substantially or completely liquid impermeable and the other carrier layer, (i.e., the second carrier layer 76 can be at least partially liquid permeable and optionally substantially or completely liquid permeable. In such an aspect, the first carrier layer 74 can be disposed toward the outer surface 32 and the second carrier layer 76 can be disposed toward the inner surface 30. As such, liquid insults may pass through the second carrier layer 76 to activate the temperature change material, and the first carrier layer 74 can slow the flow of the liquid insult from leaving the temperature change member 70 thus maximizing the temperature change that can be felt by the wearer. Alternatively, the first carrier layer 74 can be liquid permeable, and in aspects with a second carrier layer 76, both carrier layers 74 and 76 may be liquid permeable. In yet another alternative, the carrier layers 74 and 76 can each include portions that are liquid permeable and liquid impermeable. Carrier layers 74 and 76 as described above can further improve the integrity of the temperature change member 70 thereby enhancing processability, and can also aid in retaining the temperature change material within the member 70.

Suitable liquid permeable materials for the carrier layers 74 and 76 include tissue layers, nonwoven layers, or combinations thereof. In particular, materials described as suitable for use as the bodyside liner 42 may also be suitable for a liquid permeable carrier layer 74 and 76. Accordingly, a liquid permeable carrier layer 74 and 76 can also be stretchable. Likewise, materials described as suitable for use as the outercover 40 may be suitable for use as a liquid impermeable carrier layer 74 and 76. Accordingly, a liquid impermeable carrier layer 74 and 76 can also be stretchable.

The pants 20 of the various aspects of the present invention can also include a moisture barrier 98. For example, as representatively illustrated in FIGS. 3, 6-8, the moisture barrier can be disposed on the temperature change member garment facing surface 88. As described above with respect to a carrier layer 74 and 76 that at least a portion of which is liquid impermeable, the moisture barrier 98 can slow the flow of the liquid insult from leaving the temperature change member 70 thus maximizing the temperature change that can be felt by the wearer.

Accordingly, the moisture barrier 98 can be provided in the pants 20 in a variety of ways as are known in the art. For example, in aspects where the temperature change member 70 includes a carrier layer 74 and/or 76, at least a portion of the first carrier layer 74 can be liquid impermeable as described above and as a result provide a portion of the moisture barrier 98. Alternatively, a portion of the temperature change composite 72 may be treated to be substantially liquid impermeable to provide the moisture barrier 98. This can be suitable where the temperature change member 70 includes carriers layer 74 and/or 76 and in aspects where the temperature change member 70 does not include any carrier layers 74 and 76.

In still yet another alternative, a separate element such as a strip or web of liquid impermeable material, such as a polymer film, can be used to provide the moisture barrier 98 (FIGS. 3, 6-8). This aspect can be suitable where the temperature change member 70 includes a carrier layer 74 and/or 76 and in aspects where the temperature change member 70 does not include any carrier layers 74 and 76. For example, the moisture barrier 98 can be disposed between the first carrier layer 74 and the temperature change composite, or the moisture barrier 98 can be outside of the carrier layer 74 relative to the temperature change composite. A suitable material for use as a moisture barrier 98 in this instance is a 0.025 millimeter (1.0 mil) polyethylene film commercially available from Edison Plastics Company of South Plainfield, N.J.

The moisture barrier 98 can be of a variety of sizes and shapes as are known in the art. In addition, the moisture barrier can be sized to optionally extend substantially the entire length of the pants 20 in the longitudinal direction 46 (FIG. 3) for ease of manufacturing. Alternatively, the moisture barrier can be registered with the temperature change member 70 in the longitudinal direction 46 to save on material costs. That is, the moisture barrier 98 can have substantially the same length in the longitudinal direction 46 as the temperature change member 70 and the longitudinal end edges of the moisture barrier and the temperature change member can be substantially in line.

Further, the can be configured to extend substantially the entire width of the pants 20 in the lateral direction 48, or alternatively, the moisture barrier 98 can be sized in the lateral direction to be substantially the same size as the temperature change member in the lateral direction 48. In a particular aspect, the moisture barrier 98 can be configured to be a similar size and shape as the temperature change member 70 disposed on substantially the entire temperature change member garment facing surface 88 (FIG. 7). Alternatively, the moisture barrier 98 can be disposed only on portions of the temperature change member garment facing surface 88 (FIGS. 3, 6 and 8). For example, as representatively illustrated in FIGS. 3, 6 and 8, the moisture barrier 98 can be suitably zoned with at least first folded region 82; that is, the moisture barrier 98 can be positioned adjacent the first folded region 82. In addition, in aspects where the temperature change member 70 includes a plurality of folded regions, such as the first and second folded regions 82 and 84, the moisture barrier 98 can be zoned with the first and second folded regions 82 and 84.

Moreover, as representatively illustrated in FIGS. 3, 6 and 8, in aspects where the temperature change member 70 includes an unfolded region 90, the moisture barrier 98 can be positioned such that it is not adjacent the unfolded region 90. As such, the flow of the liquid insult can be slowed from leaving the temperature change member 70 in the folded regions 82 and 84 thus maximizing the temperature change that can be felt by the wearer in those regions. Accordingly, the moisture barrier 98, will be less likely to cause pooling of urine over the unfolded region 90 of the temperature change member 70 thereby allowing the pants 20 to more readily absorb urine and reduce leakage.

The temperature change material 80 of the various aspects of the present invention can include a substance that provides a temperature change when placed near the wearer and contacted with urine. The temperature change can be either an absorption or release of heat that is noticeable to the wearer. Absorption of heat by the temperature change material 80 will provide the wearer with a cool sensation, while a release of heat by the substance will provide the wearer with a warm sensation. Reference is made to U.S. Patent Application Publication 2004/0254549, published Dec. 16, 2004, in the name of Olson, et al., incorporated by reference herein, for additional information regarding the mechanism by which the temperature change sensation is accomplished. Suitably, the temperature change material 80 can be provided in particulate form for ease of processing in the described aspects.

The temperature change material 80 can be homogeneously intermixed within said matrix of fibers 78. Alternatively, the temperature change material 80 can define a distribution gradient within the thickness of the temperature change composite 72. For example, the temperature change material 80 may be intermixed within the matrix of fibers 78 in greater amounts toward the inner surface 30 of the pants. Alternatively, the temperature change material 80 may be intermixed within the matrix of fibers 78 in greater amounts toward the outer surface 30 of the pants.

The temperature change material 80 is responsive to contact with an aqueous solution such as urine to either absorb or release heat. The mechanism by which this is accomplished is dissolution of the substance in the aqueous solution, swelling of the substance in the aqueous solution, or reaction of the substance in the aqueous solution. For example, the temperature change material may include particles that have a substantial energy difference between a dissolved state and a crystalline state so that energy in the form of heat is absorbed or released to the environment upon contact with urine, or the temperature change material may release or absorb energy during swelling or reacting in an aqueous solution.

While a wide variety of substances may result in a temperature change when contacted with an aqueous solution, the selection of a particular temperature change material 80, the determination of the amount to be used and the location of the substance should be based in part on the desired temperature change. Specifically, the temperature change member 70 may suitably provide the training pants 10 with a temperature change (i.e., cooler or warmer) when wet of at least about 5 degrees C., more suitably about 10 degrees C., still more suitably about 15 degrees C. Alternatively, the temperature change member 70 can provide the pant 20 with a surface temperature change when wet of from 5 degrees C. to 15 degrees C. Surface temperature changes within this range are believed to be identifiable to some extent by children of toilet training age. More suitably the temperature change member 70 can provide the pant 20 with a surface temperature change when wet of from 5 degrees C. to 10 degrees C.

Thus, in a particular aspect, where the temperature change material is endothermic, a drop in the temperature of the product when insulted can be from about 37 degrees C. to about 25 degrees C., and further to about 22 degrees C. for improved effectiveness, particularly with a preoccupied wearer (i.e., a playing child). The temperature change can suitably last for at least 10 minutes, and more suitably for approximately 15 minutes.

By way of example, polyols such as Xylitol particles may be selected to provide a cooling sensation, as Xylitol particles absorb heat when dissolved in an aqueous solution. Alternatively, other polyols such as Sorbitol or Erithritol may be advantageously selected to provide a cooling sensation. In yet another alternative, various combinations of the above temperature change materials may be utilized.

Other suitable temperature change materials that absorb heat during dissolution include salt hydrates, such as sodium acetate (H₂0), sodium carbonate (10H₂O), sodium sulfate (10H₂O), sodium thiosulfate (5H₂O), and sodium phosphate (10H₂O); anhydrous salts such as ammonium nitrate, potassium nitrate, ammonium chloride, potassium chloride, and sodium nitrate; organic compounds such as urea and the like or combinations thereof.

The temperature change material 80 may also include those substances that absorb or release heat during swelling. By way of illustration, one suitable temperature change material that releases heat during swelling is a lightly cross-linked partially neutralized polyacrylic acid. Other temperature change material 80 that releases heat during dissolution includes aluminum chloride, aluminum sulfate, potassium aluminum sulfate, and the like or combinations thereof.

The temperature change material 80 can also include ortho esters or ketals such as menthone ketals that result from reacting menthone with alcohols containing 1 to 8 carbons or polyols containing 2 to 8 carbons, and all structural and optical isomers thereof. Particular menthone ketals that may be suitable include menthone-glycerol ketal and menthone-propylene glycol ketal. Particular ketals are disclosed in U.S. Pat. No. 5,348,750 issued to Greenberg, and U.S. Pat. No. 5,266,592 issued to Grub et al.

Thus, as described above, the temperature change member 70 can include a matrix of fibers 72 and the temperature change material 80, such as Xylitol particles, intermixed within the matrix of fibers 72. Once wet by urination, the Xylitol dissolves, creating an endothermic response, thereby signaling to the wearer that urination has occurred.

The temperature change member 70 is disposed within the training pants 20 so that, upon urination, liquid makes contact with the temperature change material 80. For example, the temperature change member 70 can be disposed with the absorbent body 44, for example intermediate the outercover 40 and liner 42. In particular, the temperature change member 70 may be attached to the absorbent body 44 and disposed toward the inner surface of the pants 20. Alternatively, the temperature change member 70 may be attached to the liner 42 adjacent the absorbent body 44. In still yet another alternative, the temperature change member 70 can be disposed within a gap between potions of the absorbent body 44 and attached, for example, to the outercover 40. Such an aspect is described in U.S. patent application Ser. No. 10/955,534 filed Sep. 29, 2004, in the name of Weber, et al., the disclosure of which is incorporated by reference to the extent that it is consistent (i.e., not in conflict) herewith.

As can be readily appreciated, the temperature change member 70 can be of various shapes and sizes. For example, the temperature change member 70 can be rectangular and can have a width in the lateral direction 48 of from 2.5 cm to 10 cm and a length in the longitudinal direction 46 of from 2.5 cm to 25 cm. In one aspect the temperature change member 70 can measure about 8 cm by about 10 cm. Alternatively, the temperature change member 70 can be oval in shape, circular, triangular, or the like. In yet another alternative, the temperature change member 70 can be generally be provided in strips that extend in the lateral 48 or longitudinal direction 46 and that can be separated by a gap.

Further, the temperature change member 70 can suitably contain various amounts of the temperature change material 80 as described above. Further, it will be understood by those of skill in the art that the training pants 20 of the present invention could include more than one temperature change member 70.

Therefore, as can be readily appreciated, the pants 20 of the various aspects of the present invention provide a temperature change member 70 that can more readily and effectively signal urination to the wearer, even when deformed or buckled in the crotch of the wearer in use.

A suitable procedure for determining the temperature change when wet of a product containing a temperature change material is described below in the temperature change test as follows. The test should be conducted in an environment having a stable temperature of 21 degrees C. to 22 degrees C. and a stable humidity of about 50 percent. The product to be tested is prepared by removing any elastic side panels and cutting all other elastics to permit the product to lay as flat as possible. The product is positioned in a Plexiglas cradle to simulate the configuration of the product in actual use. The center of the product is placed in the deepest portion of the cradle.

A liquid dispensing nozzle operatively connected to a liquid dispensing pump is positioned to dispense saline onto the inner surface of the product. The tip of the nozzle should be located 1 cm away from the inner surface and 10 cm forward of the center of the product, along the product's longitudinal axis. The pump is activated to dispense 90 ml of a stabilized isotonic 0.9 percent saline at a rate of 15 ml/sec. The saline is certified blood bank saline available from The Baxter Healthcare Corporation, Scientific Products Division, McGraw Park, Ill., and is at a temperature of 370° C.

The surface temperature of the product at the location of the temperature change member is measured using a standard thermometer or temperature sensing thermistors connected to a digital display or recording device. The surface temperature 30 seconds after the saline is dispensed is recorded as the test temperature. A reference temperature is obtained by performing this test on a portion of the product not including the temperature change material or on a similar product without the temperature change material. The surface temperature change when wet for the product is the difference between the test temperature and the reference temperature.

As various changes could be made in the above constructions and methods, without departing from the scope of the invention, it is intended that all matter contained in the above description and shown in the accompanying drawings shall be interpreted as illustrative and not in a limiting sense.

When introducing elements of the invention or the preferred aspect(s) thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. An absorbent article defining an article first side edge and an article second side edge opposite said article first side edge and a pair of end edges, said absorbent article comprising:
A liquid impermeable outercover;
An absorbent body disposed on said outercover; and
A temperature change member disposed with said absorbent body, said temperature change member comprising a temperature change member first surface and a temperature change member second surface opposite said temperature change member first surface and a temperature change material disposed between said first surface and said second surface wherein said temperature change first surface is in an overlaid relationship with itself to define a first folded region and wherein said temperature change member provides said article with a temperature change of at least 5 degrees C., as determined by the temperature change test described herein.

2. The absorbent article of claim 1 wherein said first folded region comprises a C-fold.

3. The absorbent article of claim 1 wherein said temperature change first surface is in a direct facing relationship with itself to provide said first folded region.

4. The absorbent article of claim 1 wherein said first folded region is proximate said first side edge.

5. The absorbent article of claim 1 wherein said temperature change member further defines an unfolded region.

6. The absorbent article of claim 1 wherein said temperature change member further defines a second folded region.

7. The absorbent article of claim 6 wherein said temperature change member further defines an unfolded region disposed between said first folded region and said second folded region.

8. The absorbent article of claim 6 wherein said first folded region is proximate said article first side edge and said second folded region is proximate said article second side edge.

9. The absorbent article of claim 1 wherein said first surface defines a temperature change member bodyfacing surface.

10. The absorbent article of claim 1 wherein said first surface defines a temperature change member garment facing surface.

11. The absorbent article of claim 1 wherein said temperature change member defines a bodyfacing surface and a garment facing surface opposite said bodyfacing surface and said absorbent article further comprises a moisture barrier disposed on said garment facing surface of said temperature change member.

12. The absorbent article of claim 11 wherein said moisture barrier is adjacent said first folded region.

13. The absorbent article of claim 12 wherein said temperature change member further defines an unfolded region and said moisture barrier is not adjacent said unfolded region.

14. The absorbent article of claim 1 wherein said first folded region comprises a plurality of folds.

15. The absorbent article of claim 14 wherein said first folded region comprises a Z-fold.

16. The absorbent article of claim 1 wherein said temperature change member further defines a second folded region and wherein said first folded region and said second folded region each comprise a plurality of folds.

17. The absorbent article of claim 1 further comprising an elastic member disposed with said first folded region.

18. The absorbent article of claim 17 wherein said elastic member is sandwiched within said first folded region.

19. The absorbent article of claim 1 wherein said temperature change member further comprises a coform matrix of fibers disposed between said temperature change member first surface and said temperature change member second surface, said coform matrix of fibers comprising polymer fibers and absorbent fibers, and wherein said temperature change material is intermixed within said coform matrix of fibers.

20. The absorbent article of claim 1 wherein said temperature change member defines a temperature change member area and said first folded region defines a first folded region area that is at least 33 percent of said temperature change member area.

21. The absorbent article of claim 1 wherein said temperature change material is an endothermic material.

22. The absorbent article of claim 1 wherein said temperature change material is an exothermic material.

23. The absorbent article of claim 1 wherein said temperature change material comprises xylitol.

24. The absorbent article of claim 1 wherein said temperature change material comprises sorbitol.

25. The absorbent article of claim 1 wherein said temperature change material comprises erithritol.

26. The absorbent article of claim 1, wherein the temperature change member provides the article with a surface temperature change when wet of at least 10 degrees C., as determined by the temperature change test described herein.

27. The absorbent article of claim 1, wherein the temperature change member provides said article with a surface temperature change when wet of from 5 to 15 degrees C., as determined by the temperature change test described herein.

28. An absorbent article defining an article first side edge and an article second side edge opposite said article first side edge and a pair of end edges, said absorbent article comprising:
A liquid impermeable outercover;
An absorbent body disposed on said outercover; and
A temperature change member disposed with said absorbent body, said temperature change member comprising a temperature change member first surface and a temperature change member second surface opposite said temperature change member first surface and a temperature change material disposed between said first surface and said second surface wherein said temperature change first surface is in an overlaid relationship with itself to define a first folded region, a second folded region, and an unfolded region disposed between said first folded region and said second folded region wherein said temperature change member provides said article with a temperature change of at least 5 degrees C., as determined by the temperature change test described herein.

29. The absorbent article of claim 28 wherein said first folded region is proximate said article first side edge and said second folded region is proximate said article second side edge.

30. The absorbent article of claim 28 further comprising a first elastic member disposed with said first folded region and a second elastic member disposed with said second folded region.

31. The absorbent article of claim 28, wherein the temperature change member provides said article with a surface temperature change when wet of from 5 to 15 degrees C., as determined by the temperature change test described herein.

32. An absorbent article defining an article first side edge and an article second side edge opposite said article first side edge and a pair of end edges, said absorbent article comprising:
A liquid impermeable outercover;
An absorbent body disposed on said outercover; and
A temperature change member disposed with said absorbent body, said temperature change member comprising a temperature change material and a plurality of layers in at least a portion of said temperature change member wherein said temperature change member provides said article with a temperature change of at least 5 degrees C., as determined by the temperature change test described herein.
